(19) United States
(12) Patent Application Publication
(10) Pub. No.: US 2016/0279052 A1
(43) Pub. Date: Sep. 29, 2016

(54) Compositions For Increasing The Lipid Content Of Keratinocytes

(71) Applicant: ELC Management LLC, Melville, NY (US)

(72) Inventors: Nadine Pernodet, Huntington Station, NY (US); James McCarthy, Babylon, NY (US); Edward Pelle, Valley Stream, NY (US)

(21) Appl. No.: 15/067,275

(22) Filed: Mar. 11, 2016

Related U.S. Application Data
(60) Provisional application No. 62/138,626, filed on Mar. 26, 2015.

(51) Int. Cl. A61K 8/97; A61Q 19/08

(52) U.S. Cl. CPC A61K 8/975; A61K 8/97; A61Q 19/08

(57) ABSTRACT

Contemplated are topical compositions comprising an extract of Commiphora mukul, an extract of Phoenix dactylifera, and a seaweed extract, for increasing the lipid content of keratinocytes.

### FIELD OF THE INVENTION

The present invention relates to topical compositions for treating wrinkles of the skin.

### BACKGROUND

Various approaches to improving the appearance of wrinkled skin are known. These include those that firm up or tighten the skin by increasing the thickness of the skin. So called, “plumping” compositions are intended to make the skin thicker by increasing the volume of some component of the skin. For example, products are known that target one or more of fibroblasts, adipocytes, collagen, elastin and extrafibrillar matrix in the dermis. The idea is that as the volume of the dermis increases, the surface of the skin is stretched and made more taut, and this, in turn, would cause the depth and appearance of wrinkles to diminish. Ideally, shallow wrinkles and lines may even become unnoticeable to the unaided eye.

The Commiphora mukul is a small tree of the Burseraceae family, common in regions of India and Pakistan. Extracts of the plant mass, and of a resin that the plant produces have been used in Ayurvedic medicines.

U.S. Pat. No. 5,273,747 discloses methods of extracting various fractions of Commiphora mukul, and describes their use in topical compositions for treating prostate problems, allergic dermatitis, psoriasis and inflammation due to UV radiation.

WO96/03033 discloses the use of Commiphora mukul extract as a pigmenting agent in compositions for promoting hair and skin pigmentation.

U.S. Pat. No. 5,690,948 discloses the use of a lipophilic ethyl acetate extract of Commiphora mukul as an antisebum and/or antioxidant active in cosmetic skin care compositions. The extract is said to be capable of delivering dual benefit to the skin: controlling or preventing sebum secretion, and protecting the skin from free radical damage.

U.S. Pat. No. 5,972,341 and U.S. Pat. No. 6,630,177 disclose certain extracts of Commiphora mukul that, in culture, accelerate the production of intracellular triglycerides through an effect on fibroblasts in the dermis. These same extracts, via a separate mechanism, are reported to inhibit the degradation of intracellular triglycerides, in culture. The references suggest, but do not measure or verify, that the increase of lipids in fibroblasts of the dermis (and in adipocytes into which the fibroblasts differentiate) cause in an increase in cell volume that leads to increased tonicity of the dermis, and a decrease in the depth of wrinkles.

U.S. Pat. No. 6,193,975 and US2002/0098213 identify Commiphora mukul as an agent which promotes the synthesis of the extracellular matrix of the skin.

US2004/0166178 discloses compositions comprising Commiphora mukul extract for topical application to the skin, as an agent to soften lines and/or relax the skin by an effect on muscle innervation.

WO2013/045762 discloses the combination of hydrolyzed hyaluronic acid, sodium hyaluronate and Commiphora mukul extract in a composition for volumizing the lips.

US2015/0010615 pertains to supercritical carbon dioxide extracts of Commiphora mukul resin, and methods for their use, which includes inhibiting the transformation of pre-adipocytes to adipocytes, and inhibiting triglyceride storage.

As far as the present inventors can tell, the effects of Commiphora mukul extract on the neutral lipid content of keratinocytes have not been reported. Unlike adipocytes and fibroblasts, keratinocytes reside in the epidermis, the outermost layer of the skin, while adipocytes and fibroblasts reside further down, in the dermis, which is often not reached by topically applied active agents.

Extract of date palm fruit (Phoenix dactylifera) is known to be used in topical skin care compositions, including those intended to treat wrinkles. US2005/021751 and US2010/0047361 disclose compositions comprising Phoenix dactylifera extract for increasing the concentration of active TGFb1 in the skin, for promoting the proliferation of fibroblasts in the skin, for increasing the synthesis of the constituents of the extracellular matrix (ECM) in the skin, for inhibiting degradation of the extracellular proteins of the extracellular matrix in the skin, and for exerting an anti-wrinkle or anti-ageing effect of the skin.

US2009/0264291 discloses anti-proliferative properties of aqueous extracts Phoenix dactylifera.

US2013/0011349 discloses compositions comprising date palm (Phoenix dactylifera) pollen or an extract thereof. The compositions may be formulated for topical use in methods of treatment or prophylaxis of various skin conditions or methods to counteract the effects of skin aging, including wrinkles, lines, skin dryness, dark spots, reduction in skin elasticity, increase in skin roughness.

As far as the present inventors can tell, the effects of Phoenix dactylifera extract on the neutral lipid content of keratinocytes have not been reported.

Algae extract is a known ingredient in topical skin care products, functioning as a moisturizer in all sorts of products, as well as a fragrance ingredient, emollient and skin conditioning agent. A beneficial effect on wrinkles ha been reported, but as far as the present inventors can tell, the effects of algae extract on the neutral lipid content of keratinocytes have not been reported.

### SUMMARY

Contemplated are compositions comprising an extract of Commiphora mukul, an extract of Phoenix dactylifera, a seaweed extract and a safe and stable cosmetically acceptable base. The compositions will generally be aqueous and comprise at least 50% water. The compositions may comprise any other ingredients that do no interfere with the production or retention of neutral lipids in the neutral lipid drops of keratinocytes. These compositions, when topically applied, are expected to increase the neutral lipid content of lipid drops in keratinocytes, and a relatively rapid improvement in the appearance of fine lines and wrinkles in the skin.

### DETAILED DESCRIPTION

All concentrations are by weight of the total composition unless otherwise indicated.

### Cell Culture and Neutral Lipid Detection

In many cell types, including keratinocytes, neutral lipids are concentrated in neutral lipid drops. Following treatment of keratinocytes by the agents under investigation, we stained cell samples with a dye that has specific affinity for neutral lipid drops, and measured their change in volume.

Neonatal human epidermal keratinocytes were incubated with certain extracts to determine the effect of those extracts on the lipid content of the keratinocytes. The following procedure was used.

Cell Culture—Glass-bottom, six-well plates (MatTek, Part number PO6G-1.5-20-F, Lot TKO-520-337) were coated with fibronectin (Sigma F-1141-2MG) at 25 mg/ml in D-PBS for one hour then rinsed twice with D-PBS. Thereafter, neonatal human epidermal keratinocytes (Life Technologies, lot 1391145, Passage 4) were seeded in the wells at 25,000 cells/cm². Growth medium (EpiLife® supplemented with HKGS (Life Technologies)) was added to the wells. The wells were incubated overnight at 37° C., 5% CO₂.

The next day the keratinocytes were treated with one or more actives. The actives included:
Commipheroline—a commercially available form of Commiphora mukul resin extract (90% caprylic/capric triglyceride, 10% Commiphora mukul resin extract)
Thallips—a commercially available form of seaweed extract (99% caprylic/capric triglyceride, 1% algae (seaweed) extract)
D'Orientine™ S—a commercially available form of date palm extract (95.9% caprylic/capric triglyceride, 3% Pheonix dactylifera fruit extract, 1% rapeseed sterols, 0.1% tocopherol

Cell samples were tested with each active individually, and in combinations of two, and all three together. The actives were dispersed in DMSO at concentrations of Commipheroline 2.0%, Thallips 10.0%, D'Orientine™ S 5.0%, and then each treatment was diluted 100× in growth medium, so that the final concentration of each active was commipheroline at 0.02%, Thallips at 0.1%, and D'Orientine™ S at 0.05%. The keratinocytes were exposed to the actives for 18 hours, and then the media were removed. The keratinocytes were fixed in 3.7% formaldehyde for 10-15 minutes, and then washed twice in D-PBS.

The fixed cells were stained for neutral lipid drops using HCS LipidTOX™ Red (Thermo Fisher Scientific, catalog #H34476, Lot 1605881) at 1:200, in D-PBS for 30 minutes at room temperature, in the dark. HCS LipidTOX™ Red has specific affinity for neutral lipid drops. At 25 minutes, one drop of NucBlue® Fixed Cell Stain ReadyProbes™ was applied to locate cell nuclei.

At the end of the incubation, the cells were imaged for neutral lipid drop detection on a confocal microscope (Nikon) at 60× oil immersion. Image processing was done with NIS-Elements AR and NIS-Elements C (Nikon). Z-stack imaging was done, and the lipid content was measured using object count and volume measurement on the red channel The volume of the lipid content was normalized to the number of cells as counted by the number of blue nuclei stained. Two trials were run. Results are shown in the following tables.

Percent Increase of Neutral Lipid Volume in Incubated
Human Epidermal Keratinocytes - Trial 1

| Active | % Increase |
|---|---|
| Commipheroline | 10,228 |
| Thallips | 189 |
| D'Orientine ™ S | 1,731 |
| Commipheroline + Thallips | 12,408 |
| Commipheroline + D'Orientine ™ S | 8,259 |
| Thallips + D'Orientine ™ S | 2,468 |
| Commipheroline + Thallips + | 14,138 |
| D'Orientine ™ S | |

Percent Increase of Neutral Lipid Volume in Incubated
Human Epidermal Keratinocytes - Trial 2

| Active | % Increase |
|---|---|
| Commipheroline | 1,307 |
| Thallips | 52 |
| D'Orientine ™ S | 460 |
| Commipheroline + Thallips | 1,111 |
| Commipheroline + D'Orientine ™ S | 577 |
| Thallips + D'Orientine ™ S | 796 |
| Commipheroline + Thallips + | 1,665 |
| D'Orientine ™ S | |

Through the increase in neutral lipid volume in keratinocytes, topical compositions comprising one or more of Commiphora mukul, an extract of Phoenix dactylifera and a seaweed extract are expected to provide fast plumping of the skin, and a reduction in the appearance of fines lines and wrinkles. Because they are nearer to the surface of the skin, treatment of neutral lipid drops in keratinocytes is expected to be more efficacious than topical products that target the dermis, which is usually difficult to reach through topical application.

Contemplated are compositions comprising an extract of Commiphora mukul resin (0.001%-3.0% by weight), an extract of Phoenix dactylifera (0.001%-3% by weight), a seaweed extract (0.001%-5.0% by weight) and a safe and stable cosmetically acceptable base. The compositions will generally be aqueous and comprise at least 50% water. In principle, compositions of the invention may be in any cosmetically acceptable form, i.e. emulsions, suspensions, solutions, gels, pastes, sticks, liquids suitable for aerosolized delivery, etc.

The compositions may comprise any other ingredients that do no interfere with the production or retention of neutral lipids in the neutral lipid drops of keratinocytes. This includes most known aqueous compatible skin care ingredients, in amounts that have been typical for a given agent. Such ingredients include, but are not limited to those intended to beautify the skin, benefit the skin, those intended to modify or enhance a consumer's perception of the product, and those intended to maintain the quality and integrity of the product. Examples of skin care ingredients that may be appropriate include: pigments, pearls and dyes, materials that reflect and/or refract light to alter a person's outward appearance, sunscreen, moisturizer, conditioner, exfoliators, DNA repair actives, skin barrier repair agents, anti-oxidants, proteins and other and biological additives. Wheat protein, jojoba and essential oils are just a few examples of ingredients that may be delivered to the skin in compositions of the present invention. Ingredients that tend to disrupt or destroy barrier layers of the skin are less preferred in compositions of the present invention, and are preferably avoided altogether.

Examples of ingredients that are intended to modify or enhance a consumer's perception of the product, and which may be appropriate for an aqueous phase include: fragrance, color modifiers, pH adjusters, viscosity modifiers, binders, polymers, bulking agents, film formers, plasticizers, solvents, surfactants, suspending agents, and other well known cosmetic adjuvants that modify perception.

Examples of ingredients that are intended to maintain the quality and integrity of the product, and which may be appropriate for an aqueous phase include: preservatives, emulsion stabilizers, color and odor stabilizers, buffers, chelating agents, light stabilizers, and other well known cosmetic adjuvants that maintain product integrity.

In some embodiments of the invention there may be an oil phase or a silicone phase. When dispersed in a commercial composition according to the present invention, the oil phase or silicone phase may optionally include any suitable skin benefit agent, any ingredient intended to modify or enhance a consumer's perception of the product, and/or any ingredient intended to maintain the quality and integrity of the product.

An Example of a composition according to the present invention is shown in the following table. The composition is put together with simple mixing.

| Ingredient | % by weight |
|---|---|
| water | Q.S. |
| hydroxypropyl methylcellulose (59.5%)/pullulan | 0.01 |
| (37.5%)/porphyridium cruentum extract (3.0%) | |
| bis-PEG-18 methyl ether dimethyl silane | 3.00 |
| caffeine powder | 0.20 |
| disodium EDTA | 0.05 |
| citric acid | 0.01 |
| methyl gluceth-20 | 0.50 |
| polyquaternium-51 | 1.00 |
| sodium polyacrylate starch | 0.15 |
| butylene glycol | 3.50 |
| water (97.60%)/acrylates/C10-30 alkyl acrylate | 8.00 |
| crosspolymer (2.00%)/phenoxyethanol (0.40%) | |
| glycerin | 2.00 |
| xanthan gum | 0.20 |
| caprylyl glycol | 0.40 |
| squalene (75.0%)/barley extract (15.0%)/wheat | 0.20 |
| germ extract (10.0%) | |
| propylene glycol dicaprate (54.95%)/helianthus | 1.00 |
| annus (sunflower) seed cake (40.5%)/hordeum | |
| vulgare (barley) extract (3.0%)/cucumis sativus | |
| (cucumber) fruit extract (0.75%)/ | |
| phenoxyethanol (0.8%) | |
| tocopheryl acetate | 0.25 |
| caprylic/capric triglyceride (95.0%)/laminaria | 0.30 |
| ochroleuca extract (5.0%) | |
| polysorbate 20 | 2.40 |
| coffee seed extract | 0.10 |
| boswellia serrata extract | 0.01 |
| BHT | 0.09 |
| mango seed butter | 1.00 |
| dimethicone (83%)/polysilicone-11 (17%) | 9.00 |
| dimethicone | 1.50 |
| lauryl PEG-9 polydimethylsiloxyethyl dimethicone | 2.00 |
| silica | 0.50 |
| phenoxyethanol | 0.30 |
| ethylhexylglycerin | 0.50 |
| acetyl-glucosamine | 0.25 |
| aminopropyl acsorbyl phosphate | 0.04 |
| water/sodium hydroxide 30% | 0.36 |
| sodium hyaluronate | 0.10 |
| acetyl hexapeptide-8 | 0.005 |
| whey protein | 0.10 |
| siegesbeckia orientalis extract | 0.40 |
| hydrolyzed rice extract | 0.09 |
| laminaria digitata extract | 0.01 |
| ergothioneine | 0.0002 |
| chlorella vulgaris extract | 0.005 |
| hellianthus annus seed extract | 0.006 |
| bamboo extract | 0.035 |
| pea extract | 0.05 |
| saccharide isomerate | 1.00 |
| soy protein | 0.50 |
| palmaria palmata extract | 0.008 |
| urea | 0.12 |
| algae extract | 2.00 |
| corn kernel extract | 0.02 |
| D'Orientine(TM) S^{#} | 1.00 |
| Thallips EL^{##} | 0.50 |
| Commipheroline^{###} | 0.20 |
| PEG-8 | 0.04 |
| crithmum maritimum extract | 0.01 |
| lactic acid | 0.16 |
| polyimide-1 | 0.003 |
| fragrance | 0.06 |
| acrylamide/sodum acryloyldimethyltaurate | 0.45 |
| copolymer (37.5%)/water (30%)/ | |
| isohexadecane (22.5%)/polysorbate 80 (7.5%)/ | |
| sorbitan oleate (2.5%) | |
| FD&C Red No. 4 | 0.0002 |
| tetrahydroxypropyl ethylenediamine | 0.0001 |

^{#}caprylic/capric triglyceride (95.9%)/Phoenix dactylifera (date) fruit extract (3.0%)/brassica campestris (rapeseed) sterols (1.0%)/tocopherol (0.1%)
^{##}caprylic/capric triglyceride (99.0%)/algae extract (1.0%)
^{###}caprylic/capric triglyceride (90.0%)/Commiphora mukul resin extract (10.0%)

What is claimed is:

1. A composition comprising 0.001%-3.0% by weight of an extract of Commiphora mukul resin extract, 0.001%-3.0% by weight an extract of Phoenix dactylifera, and 0.001-5.0% by weight of a seaweed extract.
